(19) United States
(12) Patent Application Publication
(10) Pub. No.: US 2022/0217970 A1
(43) Pub. Date: Jul. 14, 2022

(54) Perfusion Regulated Organ Therapeutics with Enhanced Controlled Testing

(71) Applicant: Saint Louis University, St. Louis, MO (US)

(72) Inventors: Mustafa Nazzal, Ellisville, MO (US); Ajay Kumar Jain, St. Louis, MO (US); Erik Madsen, St. Louis, MO (US); Jeffrey H. Teckman, St. Louis, MO (US)

(73) Assignee: Saint Louis University, St. Louis, MO (US)

(21) Appl. No.: 17/573,227

(22) Filed: Jan. 11, 2022

Related U.S. Application Data
(60) Provisional application No. 63/136,165, filed on Jan. 11, 2021.

(51) Int. Cl. A01N 1/02

(52) U.S. Cl. CPC A01N 1/0247

(57) ABSTRACT

The present invention relates to methods and systems that provide for a human liver perfusion model with dual blood supply channels, permitting two liver lobes split from the same donor to be pumped simultaneously for rigorously controlled therapeutic testing.

### CROSS-REFERENCE TO RELATED APPLICATION

This application claims priority to U.S. Provisional Patent Application Ser. No. 63/136,165, filed Jan. 11, 2021, and which is hereby incorporated by reference in its entirety.

### FIELD OF THE INVENTION

The present invention relates to methods and systems that provide for a human liver perfusion model with dual blood supply channels, permitting two liver lobes split from the same donor to be pumped simultaneously for rigorously controlled therapeutic testing.

### BACKGROUND OF THE INVENTION

According to the Organ Procurement and Transplant Network there were more than 13,500 patients with liver disease awaiting a transplant in 2019, while only about 8,250 livers were available for transplantation and such a trend continues in transplant medicine. Unfortunately, on average 8 people die every day while waiting for a liver transplant. Despite this great need for organs, livers are discarded from deceased donors at an alarmingly high rate. In fact, according to the Scientific Registry of Transplant Recipients, 9% of deceased donor livers were discarded and this percentage further increases for livers from donation after cardiac death (DCD).

Such livers, categorized as marginal donor livers (MDL), include those with severe macro-steatosis (>30% steatosis), elderly donors, and DCD liver. These livers are frequently discarded due to susceptibility of these organs to ischemia-reperfusion injury (IRI) which result in a range of poor outcomes from severe liver dysfunction and morbidity to primary non-function and patient death. IRI can result from cold ischemia which occurs during organ transportation or from warm ischemia either at the time of procurement in DCD donors or at the time of transplantation during the liver transplant surgery.

Several studies have evaluated methods or agents to mitigate IRI in liver transplant; however, most studies utilize cell cultures or rodent animal models limiting their translatability into clinical trials. Thus, a robust preclinical human therapeutic testing platform is needed. There has been a growing interest in normothermic perfusion (NMP) pumps for liver support which attempt to bypass cold ischemia time however these pumps remain expensive and pose logistical challenges in their transportation with the donated liver to the recipient. Moreover, they cannot eliminate IRI nor protect from warm ischemia or ischemic cholangiopathy, making them less efficacious in transplant medicine.

In light of these limitations, there has been a significant focus to develop therapeutics for MDLs to ameliorate the effects of IRI and thus enable successful transplant of MDLs. It is predicted that these therapeutics could potentially be given to the donor, used in the preservation solution, or delivered to the recipient at the time of reperfusion and increase the success rate of MDL transplants. However, major hurdles remain due to the unavailability of a reliable testing platform.

### BRIEF SUMMARY OF THE INVENTION

In one embodiment, rigorous internal control systems are developed utilizing split livers to test ferroptosis regulators modulating IRI.

In one embodiment, Perfusion Regulated Organ Therapeutics with Enhanced Controlled Testing (PROTECT) is used to pump arterial and portal blood to split human MDLs.

In one embodiment, PROTECT demonstrates therapeutic utility of ferroptosis regulators in IRI, underscoring its utility for drug discovery and rapid translatability of therapeutics.

In one aspect, a human liver perfusion system generally comprises a first container configured to hold a first human liver lobe. A second container is configured to hold a second human liver lobe. A first pump is configured to deliver a test fluid and blood to the first container. A second pump is configured to deliver control fluid and blood to the second container. A first oxygenator is in fluid communication with the first container and configured to oxygenate the blood pumped to the first container. A second oxygenator is in fluid communication with the second container and configured to oxygenate the blood pumped to the second container. A test fluid supply conduit is in fluid communication with the first container. A first blood supply conduit is in fluid communication with the first oxygenator and first container. A first fluid outlet conduit in fluid communication with the first container. A control fluid supply conduit in fluid communication with the second container. A second blood supply conduit is in fluid communication with the second oxygenator and second container. A second fluid outlet conduit is in fluid communication with the second container.

In another aspect, a method of testing a compound in a human liver perfusion model generally comprises feeding a test fluid and blood to a first human liver lobe held within a first container. Feeding a control fluid and blood to a second human liver lobe held within a second container. The method further comprises analyzing fluid and/or tissue obtained from the first human liver lobe after contact with the test fluid, and analyzing fluid and/or tissue obtained from the second human liver lobe after contact with the control fluid.

### BRIEF DESCRIPTION OF THE DRAWINGS

FIG. 1 is an illustration of a human liver perfusion system of the present disclosure;

FIG. 2 is another illustration of the human liver perfusion system;

FIG. 3A is an illustration of a right hepactic lobe with right hepactic artery and right portal vein cannulas;

FIG. 3B is an illustration of a left haptic lobe with main portal vein and common hepatic artery cannula;

FIG. 4A is a graph of ALT and AST levels over time for a first perfusion experiment;

FIG. 4B is a graph of ALT and AST levels over time for a second perfusion experiment;

FIG. 5 are photos of liver histology for the first and second perfusion experiments;

FIG. 6 are photos of immunohistochemistry for the first and second perfusion experiments;

FIGS. 7A-D are graphs of Ki67 and CK-7 quantification for the first and second perfusion experiments;

FIG. 8 are photos of iron staining for the first and second perfusion experiments;

FIG. 9A is graph of iron stain quantification for the first perfusion experiment;

FIG. 9B is graph of iron stain quantification for the second perfusion experiment;

FIG. 10A is a graph of hepatic gene expression for the first perfusion experiment; and

FIG. 10B is a graph of hepatic gene expression for the second perfusion experiment.

### DETAILED DESCRIPTION OF THE INVENTION

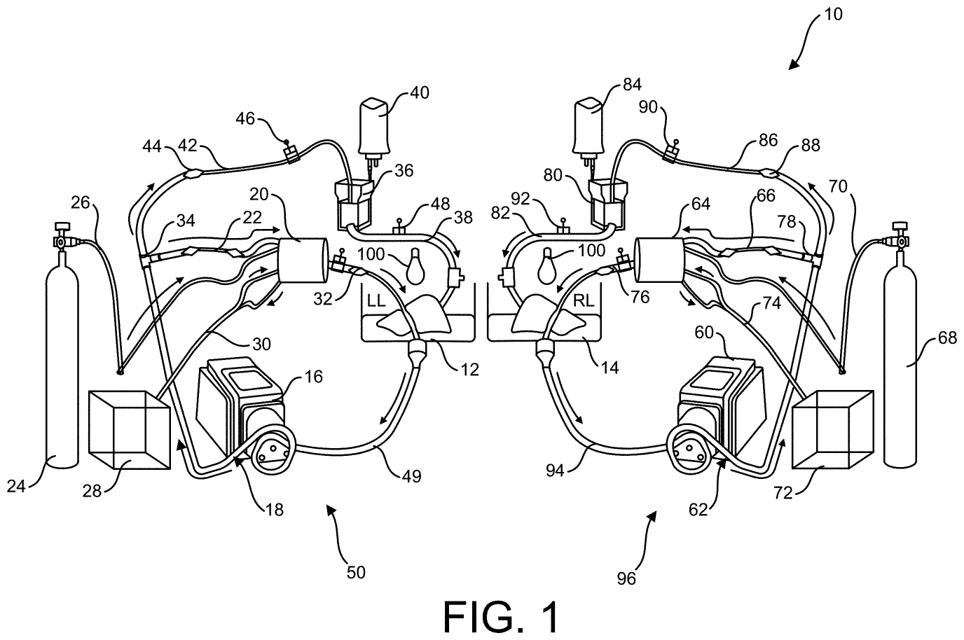

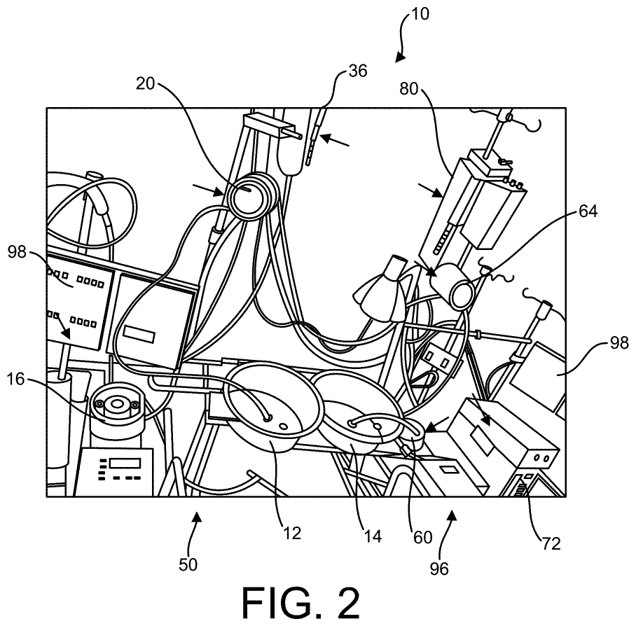

Referring to FIGS. 1 and 2, a system for human liver perfusion is generally indicated at 10. The system is configured to overcome pitfalls in current model systems. As such, in one aspect, the system 10 incorporates an improved testing platform configured with an internal control through simultaneous perfusion of both halves of a split MDL liver. Therefore, the system 10 comprises a human liver perfusion model with dual blood supply channels, permitting two liver lobes split from the same donor to be pumped simultaneously for rigorously controlled therapeutic testing. This presents a major advancement to current systems that are not equipped for simultaneous perfusion. In one embodiment, the system 10 comprises a Perfusion Regulated Organ Therapeutics with Enhanced Controlled Testing (PROTECT).

Referring to FIG. 1, the system 10 comprises a first container 12 configured to receive a first lobe LL of a human liver, and a second container 14 configured to receive a second lobe RL of a human liver. In one embodiment, the first and second lobes RL, LL comprise the split right and left lobes of a single human liver. It will be understood, however, that the containers 12, 14 could receive different lobes, such as lobes from two different livers without departing from the scope of the disclosure. A first pump 16 is configured to pump fluid to the first container 12. In the illustrated embodiment, first pump tubing 18 is mounted on the first pump 16 and extends in both an upstream and a downstream direction. The first pump 16 is configured to act on the first pump tubing 18 to drive fluid through the tubing in the downstream direction from the pump. In the illustrated embodiment, the first pump 16 comprises a peristaltic or roller pump including a plurality of rollers mounted on a rotor for rotating about a rotor axis to sequentially occlude the first pump tubing 18 driving fluid in the first pump tubing in the downstream direction.

A first oxygenator 20 is placed in fluid communication with a downstream section of the first pump tubing 18 via a first deoxygenated blood line 22. A first oxygen tank 24 is connected to the first oxygenator 20 via a first oxygen line 26, and a first heater 28 is connected to the first oxygenator by a heat conduit 30. In one embodiment, the first heater 28 comprises a water heater configured for delivering heated water through the heat conduit 30 to communicate the heated water with the first oxygenator 20 for warming the blood flowing through the oxygenator. A first oxygenated blood line 32 extends from the oxygenator 20 to the first container 12. The first oxygenator 20 is configured to warm and oxygenate the deoxygenated blood being pumped through the first pump tubing 18 by the first pump 16, as will be explained in greater detail below. In the illustrated embodiment, a connector 34 connects the first deoxygenated blood line 22 to the first pump tubing 18. However, the first deoxygenated blood line 22 could comprise a segment of the first pump tubing 18 or a segment integrally formed with the first pump tubing without departing from a scope of the disclosure. In the illustrated embodiment, the first oxygenator 20 is in fluid communication with the first container 12 but is not in fluid communication with the second container 14. In one embodiment, the first oxygenator 20 is in fluid communication with both containers 12, 14 for delivering oxygenated blood to both containers.

A first fluid reservoir 36 is disposed in fluid communication with the first container 12 by a first fluid conduit 38 extending between the first fluid reservoir and the first container. A fluid source 40 (e.g., fluid bag) configured to contain a fluid is disposed in fluid communication with the first fluid reservoir 36 for transferring the fluid from the fluid source to the first fluid reservoir. In one embodiment, the fluid in the fluid source 40 comprises a test fluid. In one embodiment, the fluid in the fluid source 40 may comprises a parenteral nutrition feeding fluid. A transition line 42 connects the first pump tubing 18 to the first fluid reservoir 36. In the illustrated embodiment, a connector 44 connects the first pump tubing 18 to transition line 42. However, it will be understood that the first pump tubing 18 could extend continuously to the first fluid reservoir 36 without departing from the scope of the disclosure. A first clamp 46 may be disposed around the transition line 42 to occlude the transition line, and a second clamp 48 may be disposed around the first fluid conduit 38 to occlude the first fluid conduit preventing fluid flow past the clamps, as will be explained in greater detail below. An upstream section 49 of the first pump tubing 18 extends from an outlet of the first container 12 to the first pump 16.

In one embodiment, the first pump tubing 18, first deoxygenated blood line 22, oxygenator 20, first oxygenated blood line 32, transition line 42, first fluid reservoir 36, first fluid conduit 38, and first container 12 comprises a first perfusion circuit 50. It will be understood that the first perfusion circuit 50 may comprise more or less components without departing from the scope of the disclosure.

The first pump 16 is configured to circulate fluid (e.g., blood, test fluid, nutrients, medication, etc.) through the first pump circuit 50 to perform perfusion treatment on the first lobe LL in the first container 12. Operation of the first pump 16 delivers fluid received from the upstream section 49 of the first pump tubing 18 through the downstream section of the first pump tubing. The fluid, and in particular, the blood in the upstream section of the first pump tubing 18 contains deoxygenated blood as it is expelled from the outlet of the first container 12. The first pump 16 forces the fluid in the first pump tubing 18 in the downstream direction toward the oxygenator 20 and first fluid reservoir 36. As such, fluid that is directed from the first pump tubing 18 through the first deoxygenated blood line 22 travels to the oxygenator 20 where the deoxygenated blood in the fluid is warmed and oxygenated. The oxygenated blood is then delivered through the oxygenated blood line 32 to the first lobe LL in the first container 12. Alternatively, fluid that is diverted away from the deoxygenated blood line 22 travels through transition line 42 to the first fluid reservoir 36. Here, test fluid, nutritional fluid, or the like, is added to the circuit 50 for delivery to the first container 12 through the first fluid conduit 38. When it is desirable to halt fluid flow from the transition line 42 and/or first fluid conduit 38 to the first container 12, the clamps 42, 48 may be engaged to occlude the fluid path from the first fluid reservoir 36 to the first container 12. Otherwise, fluid is permitted to flow freely through the transition line 42 and first fluid conduit 38 to the first container 12 to deliver the fluid to the first lobe LL in the container. Fluid in the first container 12 can then be drained through the outlet in the container and directed back to the first pump 16 for recirculation through the circuit 50.

This circulation of fluid to and from the first lobe LL in the first container 12 may function to keep the first lobe metabolically active in situations where the lobe is being treated prior to transplantation. Additionally, the supply of blood and fluid to the first lobe LL in the first container 12 can be used to facilitate analysis of fluid and/or tissue obtained from the first lobe LL after contact with the blood and fluid pumped to the first lobe. Still other processes can be performed with the first perfusion circuit 50.

Referring still to FIG. 1, the second pump 60 is configured to pump fluid to second container 14. In the illustrated embodiment, second pump tubing 62 is mounted on the second pump 60 and extends in both an upstream and a downstream direction. The second pump 60 is configured to act on the second pump tubing 62 to drive fluid through the tubing in the downstream direction from the pump. In the illustrated embodiment, the second pump 60 comprises a peristaltic or roller pump including a plurality of rollers mounted on a rotor for rotating about a rotor axis to sequentially occlude the second pump tubing 62 driving fluid in the second pump tubing in the downstream direction.

A second oxygenator 64 is placed in fluid communication with a downstream section of the second pump tubing 62 via a second deoxygenated blood line 66. A second oxygen tank 68 is connected to the second oxygenator 64 via a second oxygen line 70, and a second heater 72 is connected to the second oxygenator by a heat conduit 74. In one embodiment, the second heater 72 comprises a water heater configured for delivering heated water through the heat conduit 74 to communicate heated water with the second oxygenator 64 for warming the blood flowing through the oxygenator. A second oxygenated blood line 76 extends from the oxygenator 64 to the second container 14. The second oxygenator 64 is configured to warm and oxygenate the deoxygenated blood being pumped through the second pump tubing 62 by the second pump 60, as will be explained in greater detail below. In the illustrated embodiment, a connector 78 connects the second deoxygenated blood line 66 to the second pump tubing 62. However, the second deoxygenated blood line 66 could comprise a segment of the second pump tubing 62 or a segment integrally formed with the second pump tubing without departing from a scope of the disclosure. In the illustrated embodiment, the second oxygenator 64 is in fluid communication with the second container 14 but is not in fluid communication with the first container 12. In one embodiment, the second oxygenator 64 is in fluid communication with both containers 12, 14 for delivering oxygenated blood to both containers.

A second fluid reservoir 80 is disposed in fluid communication with the second container 14 by a second fluid conduit 82 extending between the second fluid reservoir and the second container. A fluid source 84 (e.g., fluid bag) configured to contain a fluid is disposed in fluid communication with the second fluid reservoir 80 for transferring the fluid from the fluid source to the second fluid reservoir. In one embodiment, the fluid in the fluid source 84 comprises a control fluid. In one embodiment, the fluid in the fluid source 84 may comprises a parenteral nutrition feeding fluid. A transition line 86 connects the second pump tubing 62 to the second fluid reservoir 80. In the illustrated embodiment, a connector 88 connects the second pump tubing 62 to transition line 86. However, it will be understood that the second pump tubing 62 could extend continuously to the second fluid reservoir 80 without departing from the scope of the disclosure. A first clamp 90 may be disposed around the transition line 86 to occlude the transition line, and a second clamp 92 may be disposed around the second fluid conduit 82 to occlude the second fluid conduit preventing fluid flow past the clamps, as will be explained in greater detail below. An upstream section 94 of the second pump tubing 62 extends from an outlet of the second container 14 to the second pump 60.

In one embodiment, the second pump tubing 62, second deoxygenated blood line 66, oxygenator 64, second oxygenated blood line 76, transition line 86, second fluid reservoir 80, second fluid conduit 82, and second container 14 comprises a second perfusion circuit 96. It will be understood that the second perfusion circuit 96 may comprise more or less components without departing from the scope of the disclosure.

The second pump 60 is configured to circulate fluid (e.g., blood, control fluid, nutrients, medication, etc.) through the second pump circuit 96 to perform perfusion treatment on the second lobe RL in the second container 14. Operation of the second pump 60 delivers fluid received from the upstream section 94 of the second pump tubing 18 through the downstream section of the second pump tubing. The fluid, and in particular, the blood in the upstream section of the second pump tubing 62 contains deoxygenated blood as it is expelled from the outlet of the second container 14. The second pump 60 forces the fluid in the second pump tubing 62 in the downstream direction toward the oxygenator 64 and second fluid reservoir 80. As such, fluid that is directed from the second pump tubing 62 through the second deoxygenated blood line 66 travels to the oxygenator 64 where the deoxygenated blood in the fluid is warmed and oxygenated. The oxygenated blood is then delivered through the oxygenated blood line 76 to the second lobe RL in the second container 14. Alternatively, fluid that is diverted away from the deoxygenated blood line 66 travels through transition line 86 to the second fluid reservoir 80. Here, control fluid, or the like, is added to the circuit 96 for delivery to the second container 14 through the second fluid conduit 82. When it is desirable to halt fluid flow from the transition line 86 and/or second fluid conduit 82 to the second container 14, the clamps 90, 92 may be engaged to occlude the fluid path from the second fluid reservoir 80 to the second container 14. Otherwise, fluid is permitted to flow freely through the transition line 86 and second fluid conduit 82 to the second container 14 to deliver the fluid to the second lobe RL in the container. Fluid in the second container 14 can then be drained through the outlet in the container and directed back to the second pump 60 for recirculation through the circuit 96.

This circulation of fluid to and from the second lobe RL in the second container 14 may function to maintain the second lobe in a control state in situations where the lobe is being treated prior to transplantation. Additionally, the supply of blood and fluid to the second lobe RL in the second container 14 can be used to facilitate analysis of fluid and/or tissue obtained from the second lobe RL after contact with the blood and fluid pumped to the second lobe. Still other processes can be performed with the second perfusion circuit 96.

In one embodiment, pressure monitors 98 (FIG. 2) are connected to the system 10 to monitor pressure in the first and second perfusion circuits 50, 96. In one embodiment, heat lamps 100 are mounted in relation to the first and second containers 12, 14 for warming the liver lobes LL, RL in the containers.

To demonstrate that the system 10 has a rigorous internal control both lobes LL, RL were compared using liver injury biochemical markers, histology, and gene expression. Upon sustained perfusion of split MDL lobes, simulating in vivo conditions, a similar cross-sectional and longitudinal IRI between lobes was demonstrated, thereby validating that one lobe can act as an internal control for the other. This model was then applied to a therapeutic agent in a proof-of-concept intervention to treat IRI. This process is described, in part, in the Examples section below.

### EXAMPLES

The following non-limiting examples are provided to further illustrate the present invention.

Overview

Experiment-1 compared right (UR) and left (UL) lobes to validate PROTECT.

Experiment-2 assessed ferroptosis regulator Deferoxamine in Deferoxamine Agent Treated (DMAT) vs No Agent Internal Control (NAIC) lobes. Liver serology, histology, and ferroptosis genes were assessed.

Successful MDL perfusion validated PROTECT with no ALT or AST difference between UR and UL (ΔALT_{UR}:235, ΔALT_{UL}:212; ΔAST_{UR}:576, ΔAST_{UL}:389). Liver injury markers increased in NAIC vs DMAT (ΔALT_{NAIC}:586, ΔALT_{DMAT}:−405; ΔAST_{NAIC}:617, ΔAST_{DMAT}:−380). UR and UL had similar expression of ferroptosis regulators RPL8,HO-1 and HIFα. Significantly decreased intrahepatic iron (p=0.038), HO-1 and HIFα in DMAT (HO-1_{NAIC}:6.93, HO-1_{DMAT}:274; HIFα_{NAIC}:8.67, HIFα_{DMAT}:2.60) and no hepatocellular necrosis or immunohistochemical staining (Ki67/Cytokeratin-7) differences were noted.

1. MATERIAL AND METHODS:

1.1 Liver Procurement:

Upon Institutional Review Board and Institutional Biosafety Committee approval (No. 2018-00040), human livers were procured from the Mid-America Transplant Center under an agreement with Saint Louis University. Discarded human blood was obtained from the Saint Louis University Hospital blood bank.

1.2 Graft Preparation:

Livers were procured using standardized methods of rapid flush technique developed by Miller et. al., and placed in preservation solution for transportation to the lab [8]. Ex-vivo splitting of the liver was subsequently performed while the liver was submerged in cold preservation solution.

1.3 Ex Vivo Splitting:

Anatomic dissection of the liver was conducted according to the principles described by Couinaud and Bismuth (See, Bismuth, H., Revisiting liver anatomy and terminology of hepatectomies. Ann Surg, 2013. 257(3): p. 383-6). The hilum was dissected and the common hepatic artery (CHA) identified. The CHA was subsequently dissected until the bifurcation of the right hepatic artery (RHA) and left hepatic artery (LHA). The RHA was cut and preserved with the right lobe, while the CHA and LHA were preserved for the left lobe. Then, the main portal vein (MPV) was dissected until its bifurcation. The right portal vein was cut and preserved with the right lobe graft. The MPV was preserved with the left lobe graft. The left bile duct was then dissected, cut, and preserved for the left lobe, while the common bile duct was preserved for the right lobe. Finally, the middle and left hepatic veins were cut off the inferior vena cava (IVC) and preserved with left lobe, while the IVC was preserved for the right lobe.

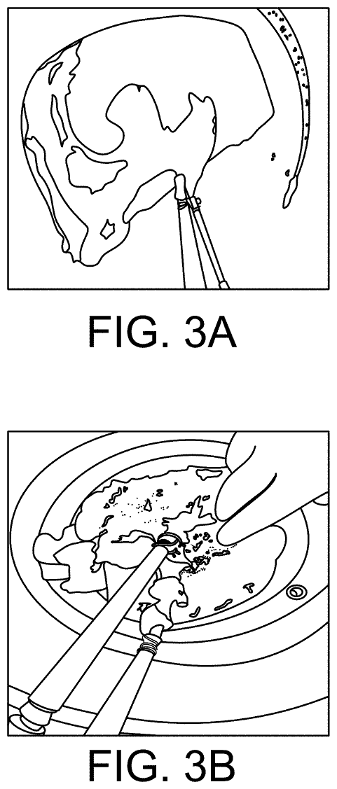

The donor liver was split along the main portal fissure separating the right and the left lobes. Utilizing crush and clamp technique, all portal vein, hepatic artery branches, and hepatic vein tributaries were ligated. Once the splitting was completed, the RHA was cannulated using an 8 Fr catheter and the RPV using an 18 Fr cannula (FIG. 3A). FIG. 3A shows a right hepatic lobe with a right hepatic artery and right portal vein cannulas. The left lobe was then cannulated using an 8 Fr cannula through the CHA and an 18 Fr cannula through the MPV (FIG. 3B). FIG. 3B shows a left hepatic lobe with a main portal vein and a common hepatic artery cannula.

1.4. PROTECT Pump:

In order to provide anatomic perfusion, a novel NMP described above and shown in FIGS. 1 and 2 was developed using an extra-corporal membrane oxygenation (ECMO) roller pump (Sorin/Stockert S3) with a modified circuit to provide dual blood supply to the hepatic artery and portal vein. Incorporated into this design was an arterial supply that passes through an oxygenator (Capiox Fx) and was connected to the hepatic artery.

Portal venous blood supply bypassed the oxygenator, was delivered to a reservoir, and then flowed by gravity into the portal vein. Pressure was regulated by the intrinsic resistance within the liver's arterial and portal venous circulations, combined with the resistance within the two limbs of the circuit and the flow delivered to each.

Flow was regulated by adjusting Hoffman clamps. Pressure monitoring was performed on both the arterial and venous limbs of the circuit: mean arterial pressure was titrated for a physiologic goal of 65-75 mmHg and a mean portal venous pressure of 10-15 mmHg. Venous drainage from the liver was collected by placing the liver in a basin with an outflow port that was connected to the intake side of the roller pump, thereby completing the circuit.

1.5 Circuit Priming and Run:

The circuit was monitored under guidance from a University certified ECMO perfusionist and the hospital ECMO physician team. The circuit was primed with a solution of 3 units of ABO-compatible packed red blood cells and 6 units of fresh frozen plasma to which 1 g calcium gluconate, 5,000 units of heparin, and 12.5 g sodium bicarbonate were added.

After de-airing the oxygenator by allowing the solution to circulate, the two limbs of the circuit were attached to the perfusion cannula which were placed into the hepatic artery and portal vein as described above and illustrated in FIGS. 3A and 3B. Flow was adjusted as described. Temperature was controlled to 36-37 degrees Celsius by use of a cardiopulmonary bypass heater attached to the oxygenator, as well as heat lamps placed near each basin.

During each perfusion run (Table 1), laboratory analysis was carried out at regular intervals. Perfusate pH, pCO₂, pO₂, bicarbonate levels, and serum electrolytes (Na⁺, K⁺, Cl⁻, ionized Ca²⁺) were monitored every hour by automated blood gas analysis of both arterial and portal venous blood samples. Adjustments to the sweep gas and blood flow were completed, as necessary. Serum markers of liver injury, specifically AST, ALT, GGT, and alkaline phosphatase, were monitored hourly. Additionally, biopsies for histologic analysis were obtained every hour from both split liver lobes.

TABLE 1
Schedule of Events

| | Monitoring | | | | |
|---|---|---|---|---|---|
| | | Portal | Arterial | Serum | |
| | | Pressure | Pressure | Chemistry | Liver |
| Event | Temp. | and Flow | and Flow | and gasses | Biopsy |
| Time 0: | Yes | No Flow | No Flow | No Flow | Yes |
| (Arrival to lab) | | Established | Established | Established | |
| Placement on | Yes | No Flow | No Flow | Yes | |
| pump (prior | | Established | Established | (In basin) | |
| to circulation | | | | | |
| Commencement | | | | | |
| of circulation: | | | | | |
| Hour 1 | Yes | Yes | Yes | Yes | Yes |
| Hour 2 | Yes | Yes | Yes | Yes | Yes |
| Hour 3 | Yes | Yes | Yes | Yes | Yes |

1.6 Study Methodology:

As part of this project two separate experiments were completed. The first, designated as Experiment 1, consisted of simultaneous perfusion of two split lobes, Untreated Right (UR) lobe and Untreated Left (UL) lobe, from a single MDL without any therapeutic interventions for a duration of three hours to validate the efficacy of the system 10. Once confirmed that both lobes were serologically and histologically equivalent in our untreated Experiment 1, the suitability of our platform for controlled therapeutic testing was assessed.

As a proof of concept, the system 10 was used to test the effect on IRI by deferoxamine (DM), which is a chelator of iron and a ferroptosis regulator. During the testing, one liver lobe was used as an internal control for the treated lobe. Ferroptosis, an iron-dependent form of programmed cell death, has been implicated as a mechanism of pathogenesis in models of liver, kidney, and lung reperfusion injury. Pertinently in the liver, in murine and cell culture models, the inhibition of ferroptosis has been shown to be therapeutic, leading to a reduction in liver damage, lipid peroxidation, and inflammatory responses. In the DM Agent Treated (DMAT) lobe, DM was administered to one split liver segment, while the other segment served as the No Agent Internal Control (NAIC) lobe. This experiment was deemed as Experiment 2.

DM Dosing: In addition to the liver lobe weight, the volume of the cold preservation solution was measured. Deferoxamine Mesylate stock (Fresenius Kabi, Lake Zurich IL) was dissolved in saline at 100 g/L. Doses were extrapolated from human chelation dosing for deferoxamine at 9 ml/kg body weight, thus providing a total chelator concentration of 26 mmol/L (iron-binding equivalents), which was achieved by DM group receiving an intravascular infusion at a concentration of 0.6 mmol/L as adapted from published data.

1.7 Histology:

Liver biopsy tissue from each liver segment was fixed in 10% buffered formalin for 24 hours. The tissue was then processed, embedded in paraffin and stained for hematoxylin and eosin (H&E). Liver tissue was also stained for Ki67 and Cytokeratin-7 (CK-7) immunohistochemistry and for iron with Perls' Prussian Blue. The automated upright microscope system with LED illumination for life sciences (Leica DM4000 B LED) was used along with Q-capture pro digital imaging software. The following describes the specific stains and the methods utilized for quantification.

Ki67 labeling index: The nuclear antigen Ki67 is expressed in the cell cycle phases G1, S, G2 and M, however not during G0. The percentage of the Ki67 positive nuclei are expressed as a labeling index, which provides an objective cell proliferation index in the tissues. Liver tissue in each group was stained via the Ki67 immunohistochemical stain. The slides were then reviewed under a light microscope by a pathologist blinded to group allocation. All the positive (immunoreactive) hepatic nuclei were calculated and subsequently divided by the total number of cells in 5 non-overlapping high-power fields. The result was reported as a Ki67 index.

CK-7 labeling index: Cytokeratin 7 is a low molecular weight cytokeratin, expressed in epithelial lining the cavities of ducts, vessels and organs. The CK-7 immunohistochemical stain is used to objectively quantify bile duct proliferation. CK-7 staining was performed on liver tissue from both segments. The slides were reviewed under a light microscope by a pathologist blinded to the group allocation. All the immunoreactive cells (cytoplasmic and membranous pattern) were calculated and reported in 5 non-overlapping high-power fields and divided by the total number of cells to compute a final CK-7 score.

Perls' Prussian Blue and Iron Quantification: Histological slides of liver samples were stained to identify non-heme iron content using Perls Prussian Blue. Slides were incubated in xylene, progressively diluted with ethanol solutions and then with distilled water. Subsequently, the slides were incubated in a potassium ferrocyanide solution to generate the blue pigment, washed, and counterstained with Nuclear Fast Red. Slides were mounted after washing with ethanol and xylene. The procedure produces blue pigment representative of iron over a light pink, cellular background.

Image J with the Fiji image processing package was used to quantify the iron staining. The Color Deconvolution tool was used on histology images to isolate the iron stain from the background stain. A mean grey value, defined as the sum of the brightness of the pixels of the iron stain divided by the number of pixels, was calculated for each image before being converted to a value that represents the product of intensity and area in fold change.

1.8 RNA extraction and real time PCR analysis:

RNA was extracted from the liver using Invitrogen™ TRIzol™ Reagent. Isolated RNA was reverse transcribed into complementary DNA using Verso cDNA Synthesis Kit (ThermoFisher, AB1453B). Primers were designed using Integrated DNA Technologies. Primers for each transcript were validated using primer blast (Table 2). Real-Time quantitative polymerase chain reaction (RT-qPCR) was performed in triplicate on the Bio-Rad CFX Connect Real-Time System. Relative mRNA levels were calculated by the comparative threshold cycle method using GAPDH as the internal control.

To describe the degree of IRI, the dimeric protein complex Hypoxia-Inducible Factor alpha (HIFα) was tested which plays an integral role in the cellular response to hypoxia. The expression of both RPL8, a mitochondrial ferroptosis modulator gene, and heme-oxygenase 1 (HO-1), a gene involved in the intracellular generation of iron were also evaluated. All these are key regulators involved in the induction of erastin-induced ferroptotic cell death and are thus reflective of the degree of ferroptosis occurring in our model.

TABLE 2
Primer Sequences

| Human Primer | | Sequence |
|---|---|---|
| GAPDH | Forward | 5′-CCATCACCATCTTCCAGGAG-3′ |
| | Reverse | 5′-GGATGATGTTCTGAGAGCC-3′ |
| RPL8 | Forward | 5′-AAGGGCATCGTAAGGACATC-3′ |
| | Reverse | 5′-CAGCTCCGTCCGCTTCTTAAA-3′ |
| HO-1 | Forward | 5′-AAGACTGCGTTCCTGCTCAAC-3′ |
| | Reverse | 5′-AAAGCCCTACAGCAACTGTCG-3′ |
| HIFα | Forward | 5′-GAACGTCGAAAAGAAAAGTCTCG-3′ |
| | Reverse | 5′-CCTTATCAAGATGCGAACTCACA-3′ |

1.9 Statistical Analysis:

Statistical analysis was performed with Graph Pad Prism version 7.03 software. Descriptive data are presented as averages, while T-tests or Mann Whitney U tests were conducted for the serological markers. Histology reads and relative mRNA expression of the genes were included where possible. All tests were two-sided using a significance level of 0.05.

2. RESULTS:

2.1 Split Livers:

Successful splitting and perfusion of MDL was achieved in two separate experiments. Temperature, pressure, and flow control were achieved in each lobe. In Experiment 1, liver injury biochemical markers, histological evaluation for hepatocellular necrosis, ductular changes, apoptosis as well as portal and lobular inflammation, immunohistochemical markers Ki-67 and CK-7, iron accumulation, and ferroptosis gene expression were evaluated with the goal of establishing the validity of the internal control. In our DM treated liver, Experiment 2, these liver injury parameters were evaluated with the objective of highlighting the ability to test therapeutics with our system 10 and detail improvement with DM.

2.2 Serological Liver Injury Markers:

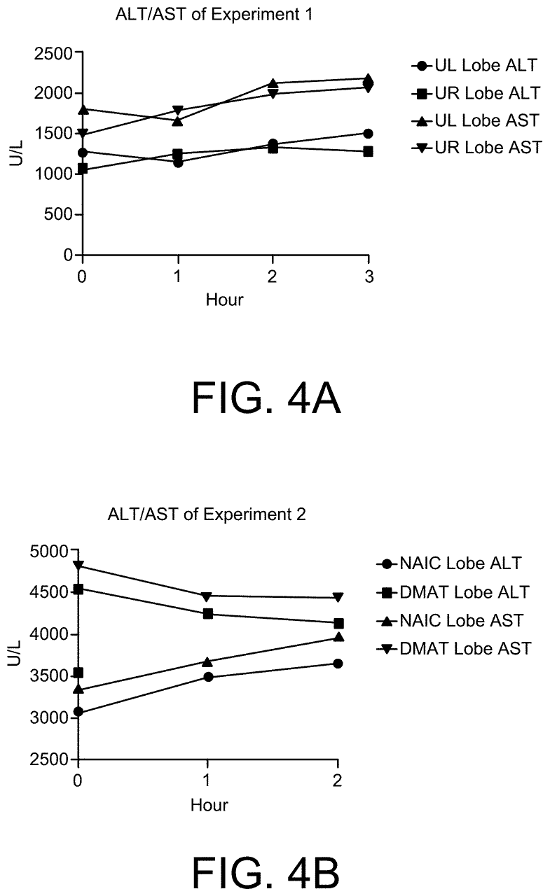

To investigate whether each lobe represented similar variability over time and thus a robust control, we measured the change in transaminase values over the course of the experiment for each MDL. In Experiment 1, ALT and AST increased similarly for both lobes (ΔALT_{UR}: 235, ΔALT_{UL}: 212; ΔAST_{UR}: 576, ΔAST^{UL}: 389). In Experiment 2, the serum ALT and AST in the DMAT lobe decreased, while the NAIC lobe's serum ALT and AST increased (ΔALT_{NAIC}: 586, ΔALT_{DMAT}: −405; ΔAST_{NAIC}: 617, ΔAST_{DMAT}: −380). FIGS. 4A and 4B demonstrates ALT and AST levels over time for each lobe during each experiment. Both GGT and alkaline phosphatase was not statistically different in Experiment 1 or Experiment 2.

2.3 Liver Histology:

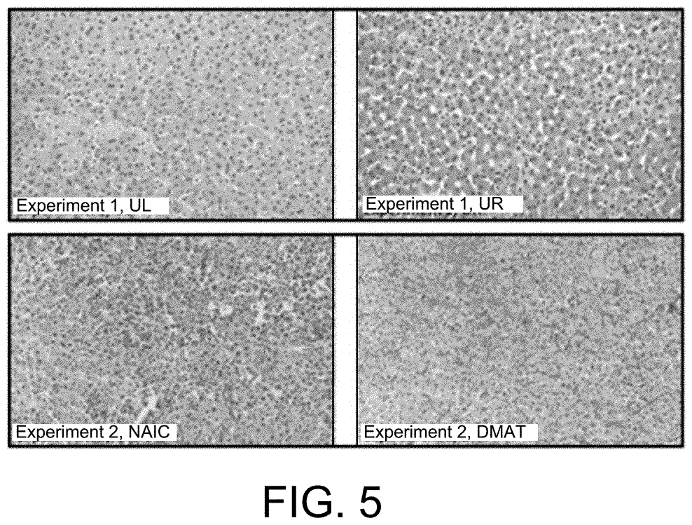

H&E: Liver histology for each experiment was evaluated objectively by a pathologist blinded to the study groups for hepatocellular necrosis, ductular changes, apoptosis as well as portal and lobular inflammation. No histological differences were noted between lobes after reperfusion cross sectionally as well as longitudinally (FIG. 5). In particular, there was no significant difference in necrosis, apoptosis, hepatocellular injury, or bile duct injury between UL and UR lobes at the end of Experiment 1. Similarly, no difference was observed between NAIC and DMAT lobes at the end of Experiment 2.

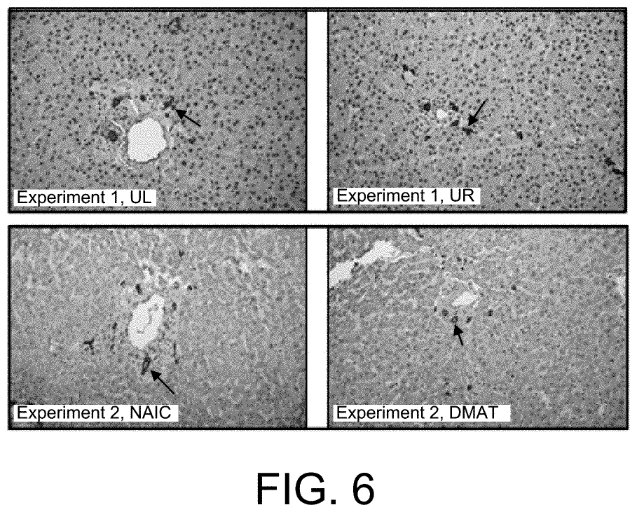

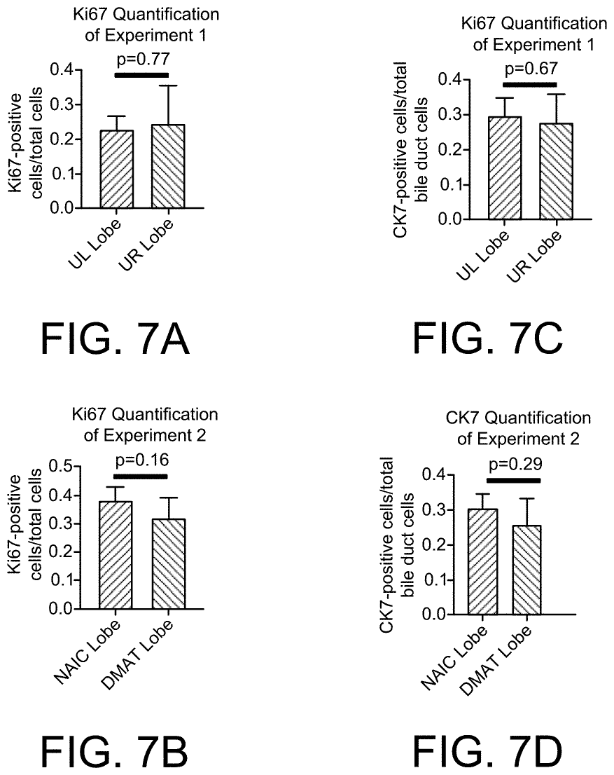

CK-7: Immunohistochemical staining for CK-7 was performed to assess for bile duct proliferation. There was no difference in CK-7 expression in either Experiment 1 (UR 0.28, UL 0.30, p=0.67) or Experiment 2 (DMAT 0.26, NAIC 0.30, p=0.29) (FIGS. 6-7D).

Ki67: Immunohistochemical staining for Ki67 was also performed on each liver lobe as a measure of hepatocellular proliferation. The Ki67 index, a ratio of proliferating to non-proliferating hepatocytes, showed no statistical inter-lobar differences at the conclusion of either Experiment 1 (UR 0.24, UL 0.23, p=0.77) or Experiment 2 (DMAT 0.32, NAIC 0.38, p=0.16) (FIGS. 7A-D). Quantification of the ration of CK7 immunoreactivity in cells surrounding portal triads was performed to evaluate bile duct proliferation. There was no difference in CK7 expression among lobes in both Experiment 1 (p=0.67, n=5) and Experiment 2 (p=0.29, n=5).

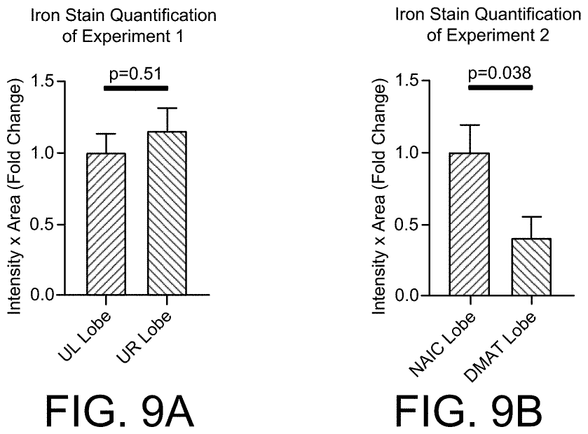

Perls' Prussian Blue: Quantification of the histological slides stained with Perls' Prussian Blue stain for iron was performed to evaluate the degree of iron chelation between liver samples at the conclusion of each experiment. Referring to FIG. 9A, in the two lobes of the split liver that were not subject to iron chelator treatment (Experiment 1), there was no difference in the degree of staining across segments (p=0.51, n=3).

However, as shown in FIG. 9B, in the second experiment, the liver segment treated with the iron chelator (Experiment 2), showed a marked decrease in iron staining compared to the untreated segment (p=0.038, n=6). This suggests that the iron chelator DM is capable of reducing intracellular iron stores in vivo. Potential implications may involve the reduction of ferroptosis within the system 10 since the process of ferroptosis is iron-dependent.

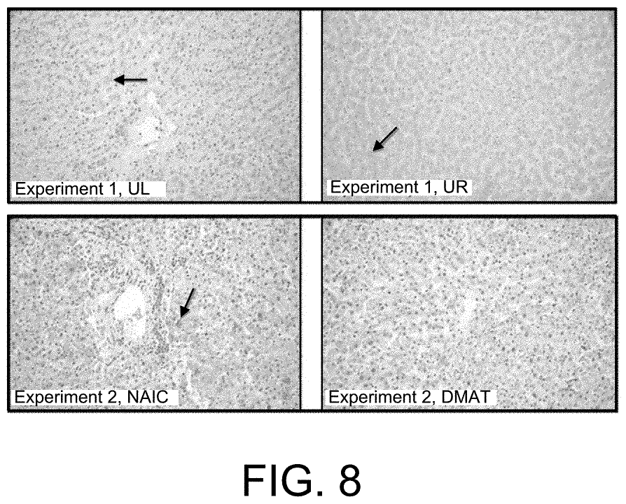

FIGS. 8 and 9 demonstrate Prussian Blue staining from Experiments 1 and 2 at the conclusion of each experiment. FIG. 8 shows histological images states for iron and imaged at the end of each experiment. There was no significant difference in iron between UR and UL lobes. There was a statistically significant reduction in iron in the DMAT lobe comparted to the NAIC lobe.

2.4 Assessment of Ferroptosis Markers

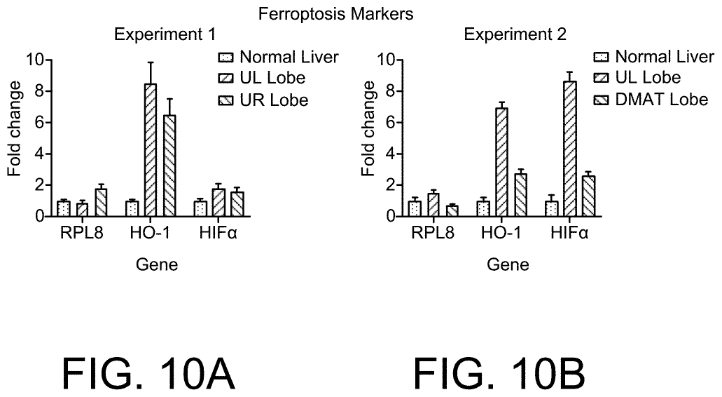

Next, HIFα, HO-1, and RPL8 expression between liver lobes was compared within Experiment 1 and 2, as well as against human liver normal control samples. In Experiment 1, RPL8 expression was higher vs normal control liver but there was minimal difference between the lobes (UR 1.80; UL 0.88) (FIG. 10A). HO-1 expression was several folds higher against the normal control liver, with no statistical difference between the two lobes of Experiment 1 (UR 6.52; UL 8.53) (FIG. 10A). HIFα was not significantly different from control nor different between the two lobes (UR 1.60; UL 1.80).

In Experiment 2, RPL8 expression was not statistically different among any of the groups (DMAT 0.72; NAIC 1.52) (FIG. 10B). However, in contrast to Experiment 1, HO-1 expression was significantly reduced upon DM treatment (DMAT 2.74; NAIC 6.93), though like in Experiment 1 both were elevated compared to the normal control liver. There was also a marked reduction in HIFα expression between the DM treated and untreated lobes (DMAT 2.60; NAIC 8.67).

3. DISCUSSION:

IRI is a major complication in liver transplantation. MDLs, which encompass human donated livers with macro-steatosis, donation after cardiac death, or liver from elderly donors, are particularly susceptible to IRI.

In order to advance strategies to expand the pool of viable livers available for transplant, a novel reperfusion model was developed for the mitigation of IRI in MDLs. The disclosed system 10, is a unique normothermic, human blood reperfusion system with dual blood supply channels. It allows two liver lobes from the same donor to be perfused concurrently, presenting an ideal unique internal control platform for the evaluation of therapeutic agents. This model serves as a preclinical method of bridging the gap between animal studies and clinical trials.

It has been shown that when each segment of an MDL is perfused under similar conditions using the system 10, they show similar liver injury transaminase values, histology, and gene expression. Pertinently, liver injury serological markers ALT and AST, revealed no significant differences between split segments. Further comparison of histology by H&E, Ki-67 labeling, CK-7 labeling, and iron labeling between split segments did not show any differences. Neither were any differences in hepatic necrosis or biliary injury, collectively suggesting equal and successful reperfusion among the segments. Analysis of IRI-related gene mRNA expression also revealed similar results between split liver lobes.

As a proof of concept, the system 10 was utilized to test the therapeutic agent deferoxamine. This iron chelator was chosen because of its ability to modulate ferroptosis as a mechanism to mitigate liver IRI. Indeed, ferroptosis is a newly described form of programmed cell death regulated by iron that is distinct from apoptosis. It is known that redox active ferrous iron, when unbound by intracellular iron storage proteins can contribute to hepatocyte death and liver damage, likely due to damage of cellular macromolecules, such as DNA, proteins, polyunsaturated fatty acids, and membrane lipids by reactive oxygen species. Arthur et al. demonstrated in their rodent model that the addition of DM to cold storage media reduced hepatocyte and endothelial cell death during cold storage, as well as liver damage on reperfusion (Arthur, P.G., et al., Desferrioxamine in warm reperfusion media decreases liver injury aggravated by cold storage. World J Gastroenterol, 2013. 19(5): p. 673-81).

Leveraging results from rodent studies, in order to evaluate the role of ferroptosis in MDL, one split lobe was treated with DM and used the other as an internal control. Transaminase levels, histology, and mRNA gene expression were evaluated between the split lobes. As noted in the results, DM treatment resulted in a decrease in AST and ALT. Pertinently, the untreated segment did not show a reduction in these liver enzymes, and, in fact, continued to show a rise in AST and ALT, indicating continued cellular damage in the untreated lobe.

Biochemical findings were confirmed by evaluating key ferroptosis markers including RPL8 and HO-1, as well as the marker of hypoxia HIF-1α. The genes from the two lobes were also compared to human liver control samples. In line with the serological results, DM treatment resulted in reduced expression of HO-1 and HIFα, indicating modulation of ferroptosis. This application of the system 10 to target ferroptosis further validates the system as a novel platform for studying IRI.

While the study validates the system 10 and suggests that therapeutics may be tested using this platform, the study was limited by a small sample size. This limitation is largely due to the scarcity of human MDLs and the inherent complexity of such a study. Another limitation was minimal liver histological differences in some of the pathological assessments as described in the results. This is attributed to the shorter experimental durations and it is predicted that these histological differences would be enhanced with longer term placement of the liver segments on the pump.

Accordingly, there are major hurdles in the development of agents to mitigate liver transplant IRI. This is a multifaceted issue due to complexity of IRI and a lack of an effective preclinical model. The disclosed system 10 simulates in vivo conditions with a robust internal control. Both segments of the liver as tested are from the same donor and thus have been subject to similar injury from pre-existing medical conditions in the donor and exposed to similar IRI. The system 10 can provide cross-sectional and longitudinal data about the effect of a variety of therapeutic agents on a human liver. The system 10 is therefore a preclinical platform for testing therapeutics. Additionally, novel data has been provided on deferoxamine as an agent to reduce IRI.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above apparatus, systems, and methods without departing from the scope of the invention, it is intended that all matter contained in the above description and shown in the accompanying drawings shall be interpreted as illustrative and not in a limiting sense.

What is claimed is:

1. A human liver perfusion system comprising:
a first container configured to hold a first human liver lobe;
a second container configured to hold a second human liver lobe;
a first pump configured to deliver a test fluid and blood to the first container;
a second pump configured to deliver control fluid and blood to the second container;
a first oxygenator in fluid communication with the first container configured to oxygenate the blood pumped to the first container;
a second oxygenator in fluid communication with the second container configured to oxygenate the blood pumped to the second container;
a test fluid supply conduit in fluid communication with the first container;
a first blood supply conduit in fluid communication with the first oxygenator and first container;
a first fluid outlet conduit in fluid communication with the first container;
a control fluid supply conduit in fluid communication with the second container;
a second blood supply conduit in fluid communication with the second oxygenator and second container; and
a second fluid outlet conduit in fluid communication with the second container.

2. The system of claim 1, wherein the first human liver lobe and second human liver lobe are split from a single human liver.

3. The system of claim 1, wherein the first container, first oxygenator, test fluid supply conduit, first blood supply conduit, and first fluid outlet conduit comprise a first perfusion circuit, and wherein the second container, second oxygenator, control fluid supply conduit, second blood supply conduit, and second fluid outlet conduit comprises a second perfusion circuit separate from the first perfusion permitting the first and second human liver lobes to be pumped with blood simultaneously for rigorously controlled therapeutic testing.

4. The system of claim 3, wherein the first perfusion circuit comprises a first pressure monitor configured to monitor pressure in the first perfusion circuit, and wherein the second perfusion circuit comprises a second pressure monitor configured to monitor pressure in the second perfusion circuit.

5. The system of claim 1, further comprising a test fluid reservoir in fluid communication with the test fluid conduit for supplying test fluid to the test fluid conduit, and a control fluid reservoir in fluid communication with the control fluid conduit for supplying control fluid to the control fluid conduit.

6. The system of claim 5, further comprising a test fluid source in fluid communication with the test fluid reservoir, and a control fluid source in fluid communication with the control fluid reservoir.

7. The system of claim 1, further comprising a first heater connected to the first oxygenator for warming the blood pumped to the first container, and a second heater connected to the second oxygenator for warming the blood pumped to the second container.

8. The system of claim 1, wherein the first and second pumps comprise rotor pumps.

9. The system of claim 1, further comprising a first clamp configured to occlude the test fluid supply conduit to restrict fluid flow to the first container, and a second clamp configured to occlude the control fluid supply conduit to restrict fluid flow to the second container.

10. The system of claim 1, further comprising a first heat lamp configured to warm the first human liver lobe when the first human liver lobe is disposed in the first container, and a second heat lamp configured to warm the second human liver lobe when the second human liver lobe is disposed in the second container.

11. A method of testing a compound in a human liver perfusion model, the method comprising:
feeding a test fluid and blood to a first human liver lobe held within a first container;
feeding a control fluid and blood to a second human liver lobe held within a second container;
analyzing fluid and/or tissue obtained from the first human liver lobe after contact with the test fluid; and
analyzing fluid and/or tissue obtained from the second human liver lobe after contact with the control fluid.

12. The method of claim 11, wherein the first and second human liver lobes are fed with blood simultaneously for rigorously controlled therapeutic testing of ferroptosis regulators modulating ischemia-reperfusion injury (IRI).

13. The method of claim 12, further comprising pumping fluid through a first perfusion circuit including the first container, a first oxygenator, a test fluid supply conduit, a first blood supply conduit, and a first fluid outlet conduit.

14. The method of claim 13, further comprising pumping fluid through a second perfusion circuit separate from the first perfusion circuit, the second perfusion circuit including the second container, a second oxygenator, a control fluid supply conduit, a second blood supply conduit, and a second fluid outlet conduit.

15. The method of claim 14, further comprising monitoring pressure in the first and second perfusion circuits.

16. The method of claim 11, further comprising oxygenating the blood fed to the first and second containers.

17. The method of claim 16, further comprising warming the blood fed to the first and second containers.

18. The method of claim 11, wherein the test fluid comprises parenteral nutrition feeding fluid.

19. The method of claim 11, wherein the first human liver lobe and second human liver lobe are split from a single human liver.
